SEQUENCE LISTING (14 sequences)

SEQ ID NO: 1 - protein, 512 aa, Oryza sativa
MAEIVSSVVVHEAVNQIIHGLVNWNERKSSAEENMERLEMAHIRLEAALETSCKWRITDSSLLCWQKKLKRAAQECDDTLRKCRQRILEQEEAEQEVRNSSIPRRIAHATKSLVSSLFYSNIDGSCRSAVRRFEWFADGANEFLRFVEFGGTPHQYLFFDPLIRQLLAGKTLEYKLVSENKYRLFVIRPFCISENRIEARLIFDSKNASALEDDFFLCMLLQVSESVDILGIVIKCLQLFNPHFMSTAESVRNELTQLPSQDFTWVPYAESCHKKHWDNIHSITTQWFRPNPLCCKQHGQNHSCESSNLGMPSVQGVSLGPVIEVSLQCHVPVPEFREQGTIVKGKPSLKKCPHMKVDLVYTPHGSSQDLLPEIKSSVIEVINGDKQHCLHTNIALELMEEIMLPRAVDCFHENAEAKLYQMLWKSKHGGAYLQVMKATMNTRSTQRTIRGSRKAKLLQQQGHKTQHRTNAISDFLNLWSAHAPVQLQGSILDWIQKEKEAQLAPPLLRLKF

SEQ ID NO: 2 - protein, 507 aa, Oryza sativa
MGEVVSSAVVHETVNKIISGLIDKYEQNSSAEEQMERLEMAHIKLETALETSSKWQITGGPLQRWQKKLKRAAEECDDTLRKCRQRVQEEEQAEQQVRNSSLPTRVAHATKSLISSIFHGNIDEPIRSAVRRFEWFADGANDFLRSVEFGGTPRRYLFFDPLIGRLLAGETLEYKLVQGNNQHLFWIRPNNTAERGVEAKLIFVYNDESVPVNNFFLGMMLQLSECTNIVGTAIKCLQLFAPHFKSTTETVRKELSQLPTQDFSWVPRSHSYHWDSIHSTVTEWFRPNPMCCKHRSQKVCSSGNMEKTYLPDISLESVIDVSLQCQVSLPGLKDQVTVVESKPSLKEFPHLKVHLVYTPHGSSEDLFPAVESSVIEMVNGVDQHCLHTNIALEQMEGIMLPRAVDCFRQNAGTTVYQMVWKSKHGGAYLQAVKVSKNMLRQRTIRGAKKAKLLRRHDHWTQSRIDVISDFLNLWAAHAPVQLQGSILDWTQKEKEAQLAPHLLHLKF

SEQ ID NO: 3 - protein, 510 aa, Oryza sativa
MGEVVSSALVHETVNKIISGMIDKYERKSSAQEHMDRLEMAQIKLDLALETSKKWQIISEPLLRWQKKLKRVAEECDDTIRMCRQRVQEEQEAKQVARDSFFPRRIAHATKSLISSIFYGNIDEPTRSTVRRFEWFADGANDFLRSVESGGTPRRYLFFDPLIGHLLAGEMLEYKLVQGNKQHLFWIRPNNIAERIEGMVFFVYNDGTAPEDNFFLGMILQISESTNIVGTIIKCLQLFAPHFESVTETVRKELTLLPTQDFSWIPHSRLYHWDNLHSIATEWFRPNPVCCKHHDQKVCGSGNMNMIELPDFSLESVIQVNLQCHVALPGFRERGTIVEGKSSLKEYLRGPHLNVLLAYTPHGSSESLFPSVEGSVIEVINANEQHCLHTNIALQQMEEIMLPRAVDYFHQNAKATVYQMLWKPKHGVAYLHAVKATVNILSTRRTIRGARKSKLLRQQDHKMHHRTDGISDFLSLWAAHAPVQLQGSILDWVQKEKEVQLAAPLLRLKF

SEQ ID NO: 4 - protein, 414 aa, Oryza sativa
MEEIVGSAIVQETVNRIISGLIDRCERKSSARDHLERLEMAQIKLEFALETSNKWQITSGPLLRWQKKLKRATEECDDTLRKCRQHIQEEDEVEQQVRNSSFPRRIAHATKTLVSSIFHSNSDELGRSSVQRFEWFADGANDFLRSVEFGGTPRRYLFFDPLIGHLISGETLEYKSIQGNKQHWFWVRPNNSAERGIEAKLFFAFNDGSAPEDNFYIDIILQLSESTNIVGTTIKCLQLFTPYFESTAETVRKELIQLPTQDFSRVSHSHSYGWENIHSIATEWFRPNPLCCKHHGQKVCGSGNLHKVELTDISLEPIIEVSLLCQVSPPGFREQGTIVEGKSSLKEFPHLNVILVYTPHGSSEDLFPAVDSTVIEVINGNEQHCLHTNVHAAKGCRLFSSECKSNSVSDALEV

SEQ ID NO: 5 - protein, 513 aa, Oryza sativa
MGEIVSSAIVTETVNKIISGMIDNYEQKLSADELMERLEMAQIKLELALETSSKWQITSEPLLRWQKKLKRATEECDDTLRKCRQHVQEEEEKEQQVRNSSFPRRIACATKSLISSIFHGNIDEPSRSTVQRFEWFAKGADDFLKSLEFGGTPRRYLFFDPLIGHLLAGETLEYKFVQGNKQHLFWIRPNDIADRGVEAKLIFVYNDCSAPENNFFLGMMLQISESTNIIGTIIKCLQWFTPHFKSTTETVRKELAQLPTQDFSWVSHYRSYHWDNIHGIATKWFRPNPICCKHQDQSMCGSGSMDKAELLDVSLQPIIEVYLERQITQFRCNSQRAAIQGKNNKQRAAVRGKRCYPRRPSHLKLGVLFLPHSSSNDLLPAAESSAVEVINGEEQPWYHRNITLEQLDKVMLPKAIGSINQNSEATAHQLLWKSKHEAAFFHLGKTRMNMPSTLSTAREATVSRRQELDLESRADVISEFLKLWVERAPVQMQRSIVDWIQKEKEVQLAPTPF

SEQ ID NO: 6 - DNA, 1539 bp, Oryza sativa
atggcagagattgttagttctgtggttgttcatgaggcagtcaaccaaatcatacatggcttggtgaactggaatgagcgaaaatcaagcgcagaggagaacatggagaggcttgagatggcacacatcaggttggaggctgcacttgagacatcctgtaagtggcgaatcactgattcttcattactgtgttggcagaagaagctgaagcgtgctgctcaagagtgcgatgacacactgcgcaaatgccggcaacgcatcctggaacaagaagaggcagaacaagaagtaaggaattcctctattcctagacgaatagcccatgccaccaagtcattagtgtcctccttattttacagcaatatcgatggctcatgtagatctgctgttcgaagatttgagtggtttgcagatggagctaatgagtttttgagatttgtggagtttggaggaacaccacatcagtacttgttctttgatcctcttatcaggcaacttcttgcaggcaaaacactagagtataaattagtgtctgaaaataagtaccggttgtttgtcatacggcccttctgtatttcagagaatagaatagaggctaggctgatctttgacagtaaaaatgctagtgcactggaggatgattttttcctgtgtatgctgctacaagtttcagagagtgtggacatacttggcattgtaatcaagtgcttgcagctgtttaatccccatttcatgtctacagctgaatctgttaggaatgaacttactcagctaccttcacaagatttcacctgggtgccatatgctgaatcatgccacaaaaaacactgggataatattcacagcattactacccaatggtttcgccctaacccattatgctgcaagcagcatggtcagaaccacagctgtgaaagtagcaatctaggaatgccatctgtacagggtgtttctctaggaccagttattgaagtgagtttgcagtgccatgtcccagttcctgagttcagagaacagggaacaattgtcaaaggcaagccttctcttaaaaaatgcccgcatatgaaagttgaccttgtctacacaccccatggctcatcacaagacctactcccagaaatcaagagttctgtaatagaggtaattaatggtgataagcaacattgcttgcacacaaacattgccttggaactaatggaagagatcatgctgccaagggcagtagattgttttcatgagaatgcagaagcgaaattgtatcagatgctttggaagtctaaacatggtggtgcatatcttcaggttatgaaggcaaccatgaacactcggagcacacagagaaccattcgaggatctaggaaagcgaagctgttgcaacagcagggtcataaaacacagcatcgtacaaatgcaatctctgacttcctcaacctttggtctgcacatgcgcctgtccagctgcagggctcaatcctcgattggattcagaaagagaaggaagcacaattagcacctcctctactgcgcctgaaattttag

SEQ ID NO: 7 - DNA, 2413 bp, Oryza sativa
gggtgaccgagagcgctggcggcgagcggcggcgccgccgccgcagccgccgcgcgcatggcggcacggggaggtaggaggcggagcatggagggggaagctagctgcgccccggcgcgacggagatgaggaggcgcaagcacaagcgcacccaccgactaccgccgcggcgagcgctcgagaaaacagaacaatgcagcaaggaggagaccaagtcatttttttgtgtctgttggtgggtctgtgtggaggtgggcatgccaagccaacataaggtgccaaatagaacaatttcaatgcatatggaagacagttcattttataattgatgctgatggaccctttcgcaaaggaagaagacttgcaatcctgtgaaacagagtgtggcaggcaatttcaccggtctgaacatatattactcaaaagttggttcagttacagaatatctggagaaataccagagctggacgcacatttatggtcttcaggcttgcagtgcaatctgtttacatcattattttcaactagtttggtgagttattgtctgctgggtactcttcggtgaacctgtgtgaccacaaattgcttcctattgattcaactcagaagggagggaacatgggagaggtagtcagttctgccgttgttcatgagacagtgaacaaaatcatatctggcttgattgacaagtatgagcaaaattcaagtgcggaggagcagatggagaggttggagatggcgcacatcaagttggagactgcacttgagacatccagtaagtggcagatcactggtgggcccttgcaacgttggcagaagaagctgaagcgtgctgctgaagagtgtgatgatacactgcgcaaatgtaggcagcgcgtccaagaagaagaacaggctgaacaacaagtaaggaattcctccttacctacgcgagttgcccatgccaccaagtcactgatatcatccatctttcacggcaatatcgacgagccgattagatctgctgttcgaagatttgaatggtttgctgatggagctaatgactttctgagatccgtggagtttggagggacaccacgtcgttacttgttctttgaccctcttatcgggcgccttcttgcaggcgaaacacttgagtacaaattagtgcaaggaaacaatcagcatttgttttggatacggccaaataacaccgcagagcgaggagtagaagctaagttgatctttgtttacaatgatgaaagtgtgcctgtgaataacttttttctaggtatgatgctacaactttcagagtgcacaaacatagttggtactgcaattaagtgcttgcagttgtttgcccctcatttcaagtctacaactgaaactgttaggaaggaacttagtcaactacctacacaggacttctcatgggtgccacgttctcattcataccattgggacagtattcacagtacagttactgaatggttccgcccaaacccaatgtgttgcaagcatcgtagtcagaaggtatgtagtagtggcaacatggagaagacatatttaccagacatttctctagaatcagttattgacgtgagtttgcagtgccaagtttcactgcctgggttaaaagatcaggtcacagttgttgagagcaaaccttctctcaaagaatttccgcatctgaaagttcaccttgtctatacgccccatggctcttcagaagacctattcccagcagtcgagagttctgtgatagagatggttaatggtgttgaccaacattgcttgcacacaaacattgccttggaacaaatggaagggatcatgctgccaagggctgtagattgttttcgtcagaatgcaggaacaacagtgtatcagatggtttggaagtccaaacatggtggtgcatatcttcaggctgtgaaggtatccaagaacatgctgaggcagagaaccattcgaggagccaagaaagcgaagctattgcgacgacatgatcattggacacagagtcgcatagatgtgatctctgatttcttaaatctttgggctgcgcatgcgcctgtccagctgcagggctcaatcctcgattggactcagaaagaaaaggaagcacagttagcacctcatctactacatctgaaattttagatcatgtaatagggacttgtcagggaaatagggatacttcaaagaatgaaaatgtactaccccgtcacaaaatataagcacttttagctaagaatatggataaaaatgcttatattttgggacaaagggagtaatattctagcaggggaagtgacttagggagttatctccaagttattaggagtgtattgatctgaaatgtgtgctccatatatttgctgctgctgtttgcgcagcgcagaacttagtaaatggtgataacttgatgttc

SEQ ID NO: 8 - DNA, 2331 bp, Oryza sativa
gcattcttgaaaactgcagtccagttatcttgtgctaggtactctctctctctgggtgcacctgtctgacccccaaggccccaagttacttcttattgattcaacccagaaggttaggccttacattgaactttgaatttctcacttttttctgtatggaatcatgcattatttcatttcactgccccctgcatgaatatctcttatatcacaaaattgattcaattcagaaggctggccttttcatatcctacagcattgcactttgttagtacttattaggaattcactggtctgtcttttgatttagtgattgatatagtatatattgattcttgatcagggagggaatcctacagattactgaagatgggggaggtggtcagttctgctctcgttcatgagacagtgaacaaaatcatatctggcatgattgacaagtatgagcgaaaatcaagtgcgcaggaacacatggacaggttagagatggcacaaatcaagttagatcttgcacttgagacatccaagaagtggcagatcattagtgagccgttgctacgttggcagaagaagctgaaacgtgttgctgaagagtgtgatgacacaatacgcatgtgcaggcagcgtgtccaggaagaacaagaggccaaacaagtagcaagggattccttctttcctaggcgaattgcccatgccaccaagtcactgatatcgtccatcttttatggcaatatcgacgagccgactagatctaccgttcgaagattcgagtggtttgcagatggagctaatgactttctgagatctgtggagtctggagggacaccgcgtcgttacttgttcttcgaccctcttattgggcaccttcttgcaggtgaaatgctagagtacaaattggttcagggaaacaagcaacatttgttttggatacggcccaataacattgcagagaggatagaaggtatggtgttcttcgtttacaatgatggcactgcacctgaggataacttttttcttggtatgatactacagatttcagagagtacaaacatagttggcactataattaagtgcttgcagttgtttgctcctcatttcgagtctgtaactgaaactgtcaggaaagaacttactctgctacctacacaagacttctcctggataccacattctcgtttataccattgggacaaccttcacagtatagctaccgaatggttccgcccaaacccagtgtgttgcaagcatcatgatcagaaggtatgtggtagtggcaacatgaacatgatagaattaccagacttctctctagaatcagttattcaagtgaatttgcagtgccatgttgcactgcctgggttcagagaacggggaacaattgtcgagggcaaatcttctctcaaagaatatctgcgtggaccgcatctgaatgttctacttgcctatacgcctcacggatcttcagaaagcctattcccatcagtcgagggttctgtgatagaggtgattaatgctaatgagcagcattgcttgcatacaaatattgccttgcaacaaatggaagagatcatgttgccgagggccgtagattattttcatcagaatgcgaaagcgacagtgtatcagatgctttggaagcctaaacatggtgttgcatatcttcatgctgtgaaggcgaccgtgaacattctgagcacacggaggaccattcgaggagctaggaaatcaaagctgttgcgacaacaggatcataagatgcatcatcgcacagatggaatctctgacttcctcagcctttgggctgcgcacgcacctgtccagcttcagggctcaatccttgattgggttcagaaagagaaggaagtacaattagcagctcctctactgcgcttgaaattttagatgatatagggctcgtttagtagagctccaactcctaaatttaactccaggagttgagtctggagtggggttatggagccgtttaaacccagctccacctctttggttcattttatgagagagttccattcagctccactcccattttgggtggagctaaaactgtttggctgagctctagctctaggagaggtggagctagtgctagtcaggaaatcagagaaacgtgaagtacaaatgtaatattttaactgagaaaagtgacttgtagggagataatatctaagtcattcggagttaactgatatgaaatgtgtgctacataaaaatgaaaactgaaaatgtcacctagtggtctgctctttcttgacctctgtaactgcttctccttctaatatatccggcagagctcctgccgtcttttgcttc

SEQ ID NO: 9 - DNA, 1977 bp, Oryza sativa
gagcgaaggctgactggctgagtagagctcgagcatgactgtactggtggagcttgagctctatctctactaaaaccaactagccaggtatgtgaatctcaatctgaaggtagagagagtgaggaagcagagctggaagaagaacactaggtcctaagtgtaacattttgtaaaaattactagcccaaagtgaaaataatagcttgttcctagtccaagctgcgaccatgggaaggattactgaagatggaagagatagtcggttctgctatcgttcaagagacggtaaacaggatcatatctgggttgattgaccggtgtgagcgaaaatcaagtgcacgggatcacctggagaggctggagatggcacaaatcaagttggagttcgcacttgagacatccaataaatggcagatcactagtggcccgttgttacgttggcagaagaagctgaagcgtgctactgaagagtgtgatgacacactacggaagtgcaggcaacatatccaggaagaagatgaggtggaacaacaagtaaggaattcttcctttcctaggcgaattgcccatgccaccaagacactggtatcttccatctttcacagcaatagtgatgagctgggtagatcttctgtccaaagattcgagtggtttgcagatggagctaatgattttctaagatctgtggaatttggaggcacaccacgccgttacttgttctttgaccctcttatcgggcaccttatttcaggtgaaacgttagagtacaaatcaatacagggaaacaagcagcattggttttgggtacggcccaataactctgcagagcgaggaatagaagctaagttgttctttgctttcaatgatggcagtgcacctgaggacaacttttacattgatattattctacaactttcagagagtacaaacatagttggtactacaattaagtgcttgcagttgtttactccttattttgagtctacagctgaaactgttaggaaagaacttattcaactacctacacaagacttctccagggtgtcacattctcactcatacggttgggagaacattcatagtatagctactgaatggttccgcccaaatccactgtgttgcaagcatcatggtcagaaagtatgtggtagtgggaacttgcacaaggtagaattaacagatatttctctagaacccatcattgaagtgagtttactgtgccaagtttcacctcctgggttcagagaacagggaacaattgttgagggtaaatcttcacttaaagaatttccgcatctgaatgttatccttgtctatacgccccatggctcttcagaagacctattcccagcagttgatagtactgtgattgaggtgattaatggtaacgagcaacattgcttgcacacaaatgttcatgctgccaagggctgtagattgttttcgtcagaatgcaaaagcaacagcgtatcagatgctttggaagtctaaacatggtggtgcatttcttcaggctgtgaaggcaacaatgaacatgcggagcacacggagaaccattcgaggagccaggaaagcaaagatgttgcgacgacatgatcgccggacacacaatcacagacatgaggtcgctgacttcctcagcctttgggctgtgcatgcacctgtccggcttcagggctcactccttgactggattcggaaggagaagaaagtagcaggtcctctactgacattttagatgatgtaatagggactagaagggaaacagggataattaaaattaaaagaatgggggagaaaagtaacatgtagggagttggtttctaagtacttaggagtgtattcatctgaaatgttctacatatactgctgttgtttgtctcgtcagttatgttggcaaatggtgataacatgatgttcttgccctagaagtctaatgtaccagccttgctcattcgatgtgtctggtttcatttg

SEQ ID NO: 10 - DNA, 2189 bp, Oryza sativa
gggagtcgctgaccaccatggcaaccaccaccgtgcacgccactgacgccatggccggacggggcctctccgccgccagatggggattgctctgccgccgaagggtccgcagcgaggccgcgccaactcgtggatcggcggcgcaaggcggtgccataatggcgtttccttcctcgatccgatgccgctgttgcttccactgtgagccgccaccatacacgcgtgcttgtgcggcgacccggcgatgcggagagggaagaaggccgccgcttcctcgcggcggccacggcgcgctagatcaagggaggcagctcttgcggattgatcactgaagatgggagagatagtcagttctgccattgttaccgagacagtgaacaaaatcatatctggcatgattgacaattatgagcaaaaattaagtgcagacgagctgatggagaggctggagatggcacaaatcaagttggagcttgcacttgagacatccagtaagtggcagatcacgagtgagccgttgctgcgttggcagaagaagctgaagcgtgctactgaagagtgtgatgacacactacgcaagtgcaggcagcatgtccaggaagaagaggagaaggaacagcaagtaaggaattcctcctttcctaggcgaattgcctgtgccaccaaatcactgatatcatccatctttcacggcaatatcgacgagccaagtagatcaactgttcaaagatttgagtggtttgctaagggagctgatgactttctgaaatctctggagtttggagggacacctcgccgttacttgttctttgaccctcttatcgggcaccttcttgctggtgaaacacttgagtacaaatttgtgcagggaaacaagcagcatttgttttggatacggcccaatgacattgcagatcgaggagtagaagctaagttgatctttgtttacaacgattgcagtgcacctgagaataactttttcctcggtatgatgctacaaatttcagagagtacaaacataattggcactataattaagtgcttgcaatggtttacccctcatttcaagtctacaactgaaactgttaggaaggaacttgctcagctccctacacaagacttctcctgggtgtcacattatcgttcataccattgggacaacattcatggtatagctactaagtggttccgcccaaacccaatatgctgcaaacatcaagatcagagcatgtgtgggagtggcagcatggacaaggcagaactgctagatgtttctcttcaaccaatcattgaagtgtatttggagcgtcaaatcacacaatttagatgcaacagtcagagggcagctatacaaggaaaaaacaataaacagagagcagctgtacgaggaaaaagatgttatccaagaaggccttcacatctgaaacttggggttctttttttgcctcatagctcttcaaatgaccttctgcctgcagctgagagttctgcagtagaggtgatcaatggcgaggagcagccttggtatcacagaaacattaccctggaacagctagacaaggtcatgctgccaaaggcaataggtagcatcaatcagaattctgaagcaacagcacaccagcttctctggaagtcaaaacatgaagctgcattctttcatttggggaagacgagaatgaatatgccgagcacattgagcactgctagggaagcaactgtgtcgagacgacaggaacttgatctggagagccgggcagatgtgatctctgagttcctcaagttgtgggttgagcgtgcgcctgtccagatgcagcgctcaattgtagactggattcagaaagagaaggaagtgcagttagcgccaacaccattctagaattctagatcatgtaagagagatcgatgagggtaacaaggggaatggataggaagttcttcatgatgagaaatgagccttgttcagtcttaatttctaagccagcaataacttggtatgtacaatagggtttcatgaaagtgaagcacagtctgttcagctcagcatataagcaagctggtaataacatggtgttgaaatattacgcaagaaatccgttcactggatgggtaatcgttctttttatctgaatttttcttgtctaatcttttgccatcattgaaagtgtgatgatgatattgatatatagttt

SEQ ID NO: 11 - DNA, 20 bp, Artificial Sequence
  ABP57-5′ primer
atggcagagattgttagttc

SEQ ID NO: 12 - DNA, 20 bp, Artificial Sequence
  RAPK1-2 primer
ctaaaatttcaggcgcagta

SEQ ID NO: 13 - DNA, 20 bp, Artificial Sequence
  ABP57-5′-1 primer
agtcggatccatggcagaga

SEQ ID NO: 14 - DNA, 20 bp, Artificial Sequence
  ABP57-3′-1 primer
agtcctcgagctaaaatttc

(19) United States
(12) Patent Application Publication
(10) Pub. No.: US 2012/0131697 A1
(43) Pub. Date: May 24, 2012

(54) GENE ENCODING AUXIN RECEPTOR PROTEIN DERIVED FROM RICE AND USE THEREOF

(75) Inventors: Dong Hern Kim, Suwon-si (KR); Keun Pyo Lee, Suwon-si (KR); Myong Il Kim, Seoul (KR); Yu Jin Kwon, Seoul (KR); Yong Sam Kim, Yuseong-gu (KR)

(73) Assignee: REPUBLIC OF KOREA (MANAGEMENT: RURAL DEVELOPMENT ADMINSTRATION), Suwon, Gyeonggi-do (KR)

(21) Appl. No.: 12/935,483

(22) PCT Filed: Jul. 21, 2010
(86) PCT No.: PCT/KR10/04754

§ 371 Date: Sep. 29, 2010

(30) Foreign Application Priority Data
Aug. 4, 2009 (KR) 10-2009-0071553

(51) Int. Cl. A01H 5/00; C12N 15/29; C12N 15/63; C12N 5/10; A01H 5/10; C12N 1/21; C12N 1/19; C07K 14/415; A01H 1/06

(52) U.S. Cl. 800/290; 530/370; 536/23.6; 435/320.1; 800/278; 800/298; 435/252.31; 435/252.33; 435/252.3; 435/252.34; 435/254.21; 435/358; 435/348; 435/366; 435/352; 435/365; 435/370; 435/357; 435/350

(57) ABSTRACT

The present invention pertains to an auxin receptor protein involved in activation of proton pump in plasma membrane of a plant derived from rice, a gene encoding the protein, a recombinant vector comprising the gene, a host cell transformed with the recombinant vector, a method of improving traits of a plant by transforming the plant with the recombinant plant expression vector, a plant having improved traits by transformation with the recombinant plant expression vector and seeds of the plant, and a composition comprising the gene of the invention for improving traits of a plant.

### CLAIM OF PRIORITY

This application makes reference to, incorporates the same herein, and claims all benefits accruing under 35 U.S.C. §119 from an application earlier filed in the Korean Intellectual Property Office on 4 Aug. 2009 and there duly assigned Serial No. 10-2009-0071553. This application also makes reference to, incorporates the same herein, and claims all benefits accruing under 35 U.S.C. §365(c) of my PCT International application entitled GENE ENCODING AUXIN RECEPTOR PROTEIN DERIVED FROM RICE AND USE THEREO filed on 21 Jul. 2010 and duly assigned Serial No. PCT/KR2010/004754.

### BACKGROUND OF THE INVENTION

1. Field of the Invention

The present invention is related to a gene encoding auxin receptor protein derived from rice, that is involved in activation of a proton pump in plant plasma membrane related to agricultural properties of various crops, and use thereof. More specifically, the present invention is related to auxin receptor protein ABP57 derived from crops like rice, corn, and etc., gene and analogous gene thereof, and their use for the improvement of traits of a plant that are related to the activity of a proton pump of a plant like growth promotion, etc.

2. Description of the Related Art

Auxin is a plant hormone which is involved in various traits of a plant. Hormone's activity of controlling physiology is achieved by a binding of a hormone to a receptor protein and a series of signal transduction pathways that are derived therefrom. Thus, characterization of a hormone receptor is the most basic requirement to understand the control mechanism of a hormone (Badescu and Napier (2006) Trends Plant Sci 11, 217-223). Studies to identify the receptor for auxin, which is one of the basic plant hormones, have been carried out for a long time, and as a result it was reported that the proteins like ABP1, TIR1, and ABP57, etc. are able to function as an auxin receptor (Hertel et al. (1972) Planta 107, 325-340).

Regarding ABP1 which was first separated from cotyledons of a corn, there are accumulated studies on separation of a gene for the 22 kD protein, binding of the protein to auxin, and physiological function, etc., and consequently it was found that ABP1 is related to the physiological effect of auxin. However, as the IAA dissociation constant of ABP1 is relatively high and the auxin signal transduction pathway after binding with auxin cannot be explained, it remains uncertain whether or not ABP1 is truly a receptor for auxin. Meanwhile, T1R1, which is identified through a series of studies on a group of genes which show different expression under the treatment with auxin hormone, was found to be a receptor for auxin hormone that is involved in the turnover of the protein degradation. However, except the regulation of the expression of auxin gene, not all of the various controlling activities of auxin hormone, for example the physiological activity of auxin including acidification of a cell wall by auxin and bell-like dose response of auxin, etc., can be explained by T1R1, and it remains as a problem.

According to the previous studies, inventors of the present invention identified the water-soluble protein from young seedlings and roots of a rice plant, which is directly related to the activation of a proton pump in plant plasma membrane by auxin (Kim et al. (1997) FEBS Lett 409, 273-276; Kim et al. (1998) FEBS Lett 438, 241-244; Kim et al. (2000) Plant Growth Regul 32, 143-150; Kim et al. (2001) J Biol Chem 276, 10730-10736). The size of the protein determined by the inventors was 57 kD, and based on the characteristics that are found in an auxin receptor like activation of a proton pump by a protein-protein interaction, IAA binding constant, and the biochemical property of directly mediating bell-like IAA-dose response which affects proton pump activation and also in view of the size of the protein, it was named “ABP57.”

### SUMMARY OF THE INVENTION

### Problems to be Solved by the Invention

The present invention is devised in view of the needs described above. The inventors of the present invention newly isolated the gene of auxin receptor ABP57 that is related to activation of a proton pump in a plasma membrane of a plant, and found out that the gene can be used for improvement of traits that are useful in agriculture like growth and development of a plant, etc., and therefore completed the invention.

### Technical Means for Solving the Problems

In order to solve the problems described above, the present invention provides an auxin receptor protein which is related to activation of a proton pump in plasma membrane of a plant derived from rice.

Furthermore, the present invention provides a gene which encodes the protein.

Furthermore, the present invention provides a recombinant vector comprising said gene.

Furthermore, the present invention provides a host cell which is transformed with said recombinant vector.

Furthermore, the present invention provides a method of improving traits of a plant by transforming the plant with said recombinant plant expression vector.

Furthermore, the present invention provides a transformed plant having improved traits by transformation with said recombinant plant expression vector and seeds of the plant.

Still furthermore, the present invention provides a composition comprising said gene for improving traits of a plant.

### Advantageous effect of the invention

According to the present invention, gene for auxin receptor ABP57 was newly identified, and according to an in vitro test and an in vivo test by which a transformant was tested in a plant, it is suggested for the first time that ABP57 gene has an ability of activating a proton pump in a plant plasma membrane and binding to auxin hormone IAA, and it is involved in plant growth which is related to the physiological function of auxin. Considering various physiological and biochemical functions of a proton pump in plant plasma membrane, with the novel gene identified by the present invention, various industrial applications like development of an agent for promoting growth of a crop plant or an agent for protecting a crop plant, etc. are expected.

### BRIEF DESCRIPTION OF THE DRAWINGS

A more complete appreciation of the invention, and many of the attendant advantages thereof, will be readily apparent as the same becomes better understood by reference to the following detailed description when considered in conjunction with the accompanying drawings, in which like reference symbols indicate the same or similar components, wherein:

FIG. 1 shows the SDS-PAGE electrophoresis of purified auxin receptor protein ABP57 that is derived from young seedlings of a rice plant and the results of amino acid sequencing of the peptide fragment that is obtained from trypsin digestion.

FIG. 2 shows the ORF amino acid sequence of ABP57 gene which is obtained by BLASTP analysis using the NCBI's protein database and the amino acid sequence information of the peptide fragment.

FIG. 3 shows the electrophoresis of the total RNA derived from rice seedlings (lane 1) and gene fragment of ABP57 that is amplified from genomic DNA (lane 2), together with the characteristics of the presumed protein that is encoded by a group of genes that are analogous to ABP57, as obtained by BLASTN analysis using full length cDNA database of a rice plant (KOME).

FIG. 4 shows the result of the comparison of amino acid sequence of the proteins that are encoded by a group of genes analogous to ABP57.

FIG. 5 shows the result of expression analysis of a group of genes analogous to ABP57 that are obtained from callus, stem and roots of rice based on microarray analysis data.

FIG. 6 shows a process of purifying ABP57 based on production of MBP (maltose binding protein)-fused ABP using E. coli system and a column.

FIG. 7 shows the assay result of proton pump in plasma membrane of a rice plant by using recombinant ABP57.

FIG. 8 shows the assay result of the interaction between the proton pump and ABP57 based on Hill Plot analysis in which rice plasma membrane activation was determined in terms of the dependency on ABP57 concentration. rABP57: recombinant ABP57 protein; nABP57: natural ABP57 protein.

FIG. 9 shows the assay result of the ABP57's binding property for IAA (indole-3-acetic acid) based on Scatchard plot analysis. rABP57: recombinant ABP57 protein; nABP57: natural ABP57 protein.

FIG. 10 shows a schematic diagram of a vector for transforming Arabidopsis thaliana with ABP57 gene and the result of gene expression analysis for the transformant.

FIG. 11 shows a photograph (A) and a graph (B) in which plant growth properties are compared between the Arabidopsis thaliana transformed with ABP57 and the control group.

FIG. 12 shows a photograph in which germination properties are compared between the rice plant (over-expression strain) transformed with ABP57 gene and the control group (Nakdong variety).

### DETAILED DESCRIPTION OF THE INVENTION

In order to achieve the object of the invention described above, the present invention provides the auxin receptor protein which is involved in the activation of a proton pump (H⁺-ATPase) in plasma membrane of a plant derived from rice, wherein the protein has an amino acid sequence selected from the group consisting of an amino acid sequence of SEQ ID NO: 1 to 5. The amino acid sequence of SEQ ID NO: 1 to 5 has Protein Id of BAC10824 (ABP57), BAC10825, BAC79737, BAC79739 and BAC83785, respectively. Preferably, the auxin receptor protein is ABP57 protein consisting of the amino acid sequence of SEQ ID NO: 1.

Included in the scope of the auxin receptor protein of the present invention is a protein having an amino acid sequence of SEQ ID NO: 1 to 5 that is isolated from rice (Oryza sativa), and functional equivalents of said proteins. The term “functional equivalent” means that, as a result of addition, substitution or deletion of amino acid residues, it has an amino acid sequence with at least 70%, preferably at least 80%, more preferably at least 90%, still more preferably at least 95% homology with the amino acid sequence that of SEQ ID NO: 1 to 5, thus indicating a protein which has substantially the same physiological activity as the protein of SEQ ID NO: 1 to 5.

Further, the present invention provides a gene which encodes the auxin receptor protein. The gene of the present invention includes both genomic DNA and cDNA which encode the auxin receptor protein. Preferably, the gene of the present invention may comprise a nucleotide sequence of SEQ ID NO: 6 to 10. The nucleotide sequence of SEQ ID NO: 6 to 10 has a cDNA Id of AK072975, AK073714, AK102706, AK102508 and AK103212, respectively. Preferably, the gene of the present invention is AK072975 consisting of the nucleotide sequence of SEQ ID NO: 6. Variants of said nucleotide sequence are also within the scope of the present invention. Specifically, said gene may comprise a nucleotide sequence with at least 70%, preferably at least 80%, more preferably at least 90%, still more preferably at least 95% homology with the nucleotide sequences of SEQ ID NO: 6 to 10. The “sequence homology %” for a certain polynucleotide is determined by comparing two nucleotide sequences that are optimally arranged with a region to be compared. In this regard, a part of the polynucleotide sequence in a region to be compared may comprise an addition or a deletion (i.e., a gap) compared to a reference sequence (without any addition or deletion) relative to the optimized arrangement of the two sequences.

The inventors of the present invention purified the auxin receptor ABP57 from the stems of rice seedling, and by digesting the purified protein with trypsin and analyzing it with ESI-MS/MS, amino acid information was obtained for the four peptides. Using non-redundant protein sequence database of NCBI, BLAST search was carried out for the amino acid sequence of the four peptides. As a result, a gene (AK072975) of which function has not been known yet was identified. With the registered nucleotide sequence of the gene, PCR primer was designed, and total RNA and genomic DNA from rice seedlings were amplified to obtain a gene fragment. As a result of the nucleotide sequence analysis, it shows 100% match with AK072975. ORF of the gene consists of 1,539 by and encodes the protein of 58.7kDa (pI 7.97) which consists of 512 amino acids.

By using the information of the nucleotide sequence of a gene above, full-length cDNA database search with KOME (Knowledge-based Oryza Molecular Biological Encyclopedia) and EST database search with Maize Genome Sequence Consortium were carried out. As a result, in addition to AK072975, four kinds of analogous genes (AK073714, AK102508, AK102706 and AK103212) with unknown function were identified from a rice plant, and ORF of each gene shows 53 to 73% of amino acid homology, respectively. Further, one gene with sequence homology to maize and unknown function was also identified. As a result of rice 60 k microarray analysis, AK072975 and the analogous genes were found to be expressed in callus, stem and roots of a rice plant.

In order to confirm whether or not the protein encoded by the gene described above has an ability of activating a proton pump, a protein expression vector comprising the isolated gene was constructed, expressed in E. coli, and then the recombinant protein was purified. The recombinant protein was tested as a sample to determine an ability of activating a proton pump in rice plasma membrane and an ability of binding to auxin. As a result, it was found that the recombinant protein has all the characteristics, and therefore identified as a gene which encodes ABP57.

Further, the present invention provides a recombinant vector comprising the gene encoding the auxin receptor protein of the invention. Preferably, the recombinant vector is a recombinant plant expression vector.

The term “recombinant” indicates a cell which replicates a heterogeneous nucleotide or expresses said nucleotide, a peptide, a heterogeneous peptide, or a protein encoded by a heterogeneous nucleotide. Recombinant cell can express a gene or a gene fragment in a form of a sense or antisense, that are not found in natural state of cell. In addition, a recombinant cell can express a gene that is found in natural state, provided that said gene is modified and re-introduced into the cell by an artificial means.

The term “vector” is used herein to refer DNA fragment (s) and nucleotide molecules that are delivered to a cell. Vector can replicate DNA and be independently reproduced in a host cell. The terms “delivery system” and “vector” are often interchangeably used. The term “expression vector” means a recombinant DNA molecule comprising a desired coding sequence and other appropriate nucleotide sequences that are essential for the expression of the operatively-linked coding sequence in a specific host organism. The promoter, enhancer, termination signal and polymerization signal which can be used by eukaryotic cells are well known in the art.

A preferred example of plant expression vector is Ti-plasmid vector which can transfer a part of itself, i.e., so called T-region, to a plant cell when the vector is present in an appropriate host such as Agrobacterium tumefaciens. Other types of Ti-plasmid vector (see, EP 0 116 718 B1) are currently used for transferring a hybrid gene to protoplasts that can produce a new plant by appropriately inserting a plant cell or hybrid DNA to a genome of a plant. Especially preferred form of Ti-plasmid vector is a so-called binary vector which has been disclosed in EP 0 120 516 B1 and U.S. Pat. No. 4,940,838. Other vector that can be used for introducing the DNA of the present invention to a host plant can be selected from a double-stranded plant virus (e.g., CaMV), a single-stranded plant virus, and a viral vector which can be originated from Gemini virus, etc., for example a non-complete plant viral vector. Use of said vector can be advantageous especially when a plant host cannot be appropriately transformed.

Expression vector may comprise at least one selective marker. Said selective marker is a nucleotide sequence having a property that can make a target gene get selected by a common chemical method. Example includes, a gene resistant to herbicide such as glyphosate and phosphinotricine, and a gene resistant to antibiotics such as kanamycin, G418, bleomycin, hygromycin, and chloramphenicol, but not limited thereto.

According to the expression vector of the present invention, the promoter can be CaMV 35S promoter, actin promoter, ubiquitin promoter, pEMU promoter, MAS promoter, or histone promoter, but not limited thereto. The term “promoter” indicates a region of DNA located upstream of a structure gene, and it corresponds to a DNA molecule to which an RNA polymerase binds to initiate transcription. The term “plant promoter” indicates the promoter that can initiate transcription in a plant cell. The term “constitutive promoter” indicates the promoter that is active under most environmental conditions and cell growth or differentiation state. Since selection of a transformant can be made for various tissues at various stages, the constitutive promoter may be preferred for the present invention. Thus, selection property is not limited by a constitutive promoter.

In the above-described recombinant vector of the invention, any kind of a typical terminator can be used. Example includes, nopalin synthase (NOS), rice α-amylase RAmy1 A terminator, phaseoline terminator, and a terminator for Octopine gene of Agrobacterium tumefaciens, etc., but are not limited thereto. Regarding the necessity of terminator, it is generally known that such region can increase a reliability and an efficiency of transcription in plant cells. Therefore, the use of terminator is highly preferable in view of the context of the present invention.

Further, the present invention provides a host cell that is transformed with the recombinant vector of the present invention. Any kind of a host cell known in the pertinent art can be used if stable and continuous cloning and expression of the vector of the present invention can be achieved by using it. Examples include strains belonging to the genus Bascillus such as E. coli JM109, E. coli BL21, E. coli RR1, E. coli LE392, E. coli B, E. coli X 1776, E. coli W3110, Bascillus subtilus, Bascillus thuringiensis, and the like, Salmonella typhimurium, intestinal flora and strains such as Serratia marcescens and various Pseudomonas Spp. and the like.

In addition, when the vector of the present invention is transformed in an eukaryotic cell, a host cell such as Saccharomyce cerevisiae, an insect cell, a human cell (e.g., CHO cell line (Chinese hamster ovary), W138, BHK, COS-7, 293, HepG2, 3T3, RIN and MDCK cell line), a plant cell line and the like can be used. Preferably, the host cell is a plant cell.

When a host cell is a prokaryotic cell, transfer of the vector of the present invention into a host cell can be carried out according to CaCl₂ method, Hanahan's method (Hanahan, D., J. Mol. Biol., 166:557-580 (1983)), and an electroporation method, etc. In addition, when a host cell is an eukaryotic cell, the vector of the present invention can be transferred into a host cell according to a microscopic injection method, calcium phosphate precipitation method, an electroporation method, a liposome-mediated transformation, DEAE-dextran treatment method and a gene bombardment method, etc.

Further, the present invention provides a method of improving the traits of a plant, comprising transforming a plant cell with the recombinant plant expression vector of the present invention. The improvement of the traits of a plant can be promotion of plant growth, but not limited thereto.

Determination of a physiological activity of a gene using an in vivo system is very important. In this connection, the inventors of the present invention over-expressed the ABP57 gene in Arabidopsis thaliana as a model plant, and observed any characteristics of the growth. As a result, it was found that the growth was at least two times faster than the control plant. Considering the in vitro test relating to the activation of a proton pump, it is believed that the growth promotion by ABP57 gene as expressed in Arabidopsis thaliana is closely related to the activation of a proton pump present in the plasma membrane of Arabidopsis thaliana.

Further, the present invention provides a plant with improved traits as transformed with the recombinant plant expression vector of the invention. The plant is preferably a plant with promoted growth. The plant includes monocot and dicot plants. Examples of monocot plant include rice, wheat, barley, bamboo shoot, corn, taro, asparagus, onion, garlic, scallion, leek, wild rocambole, hemp, and ginger, but not limited thereto. Examples of dicot plant include, tobacco, Arabidopsis thaliana, eggplant, pepper, tomato, potato, burdock, crown daisy, lettuce, Chinese bellflower, chard, spinach, sweet potato, celery, carrot, coriander, parsley, Chinese cabbage, cabbage, leaf mustard, radish, watermelon, melon, cucumber, zucchini, gourd, strawberry, soy bean, mung bean, kidney bean, sweet pea and the like, but not limited thereto. The plant is preferably Arabidopsis thaliana or rice.

Further, the present invention provides a seed obtained from the plant having improved traits by the transformation with the recombinant plant expression vector of the invention.

Still further, the present invention provides a composition for improving traits of a plant comprising the gene of the invention. According to the composition of the invention, it is preferable that the gene consists of the nucleotide sequence of SEQ ID NO: 6 to 10. It is more preferable that the gene consists of the nucleotide sequence of SEQ ID NO: 6. Preferably, the improvement of traits of a plant is a promotion of plant growth.

The present invention will now be described in greater detail with reference to the following examples. However, it is only to specifically exemplify the present invention and in no case the scope of the present invention is construed to be limited by these examples.

### EXAMPLES

### Example 1

### Amplification and Nucleotide Sequence Determination of ABP57 Gene by RT-PCR Method

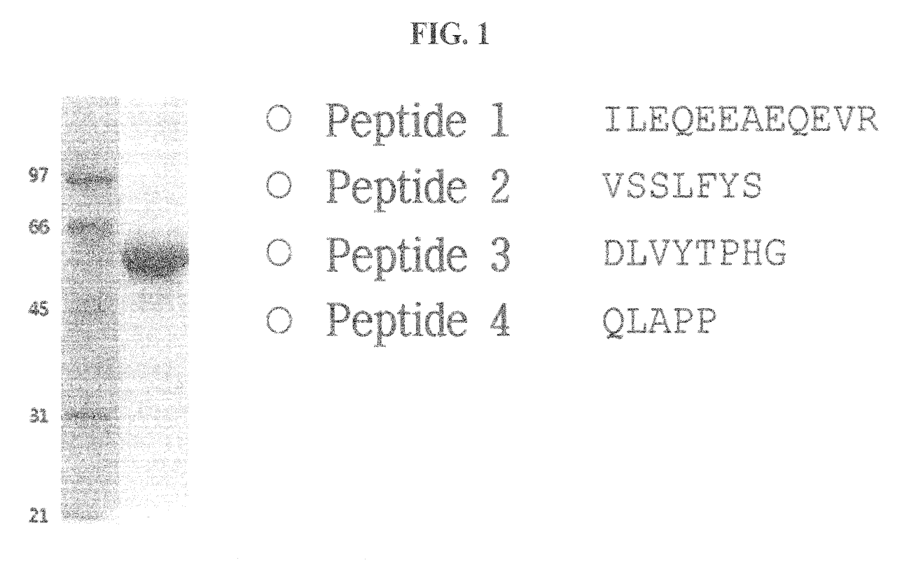

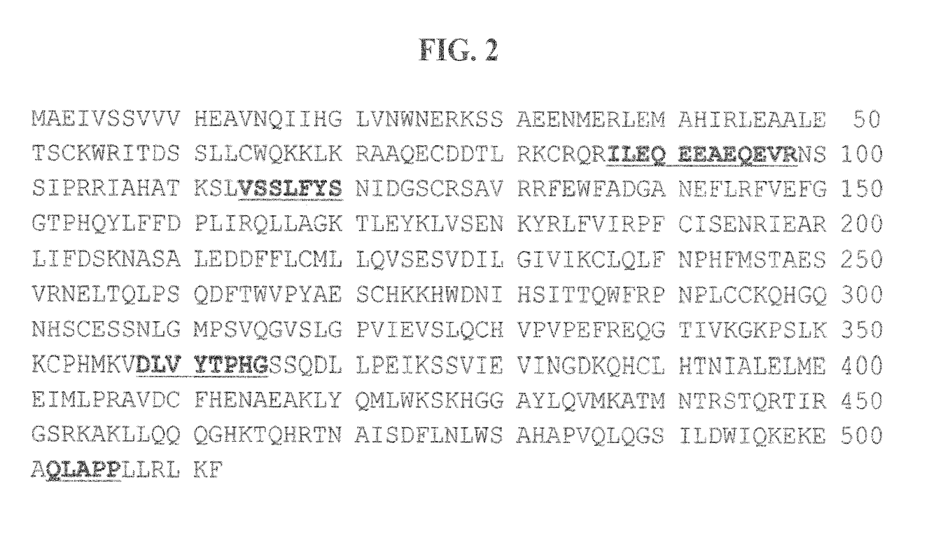

By using DEAE, CM ion exchange chromatography and tryptophan affinity chromatography, ABP567 was purified from the extract of rice seedlings of Nakdong variety. The protein was digested with trypsin, and the amino acid sequence of the digested protein was analyzed by ESI-MS/MS. As a result, the amino acid sequence information of four peptides was obtained (FIG. 1). As a result of the search of the amino acid sequence information of the peptide against NCBI's protein sequence database, one gene comprising all four peptides was found (AK072975), and the PCR primers were prepared based on the nucleotide sequence information of the gene (FIG. 2).

| | |
|---|---|
| Forward primer (ABP57-5′): | |
| 5′-ATGGCAGAGATTGTTAGTTC-3′ | (SEQ ID NO: 11) |
| Reverse primer (RAPK1-2): | |
| 5′-CTAAAATTTCAGGCGCAGTA-3′ | (SEQ ID NO: 12) |

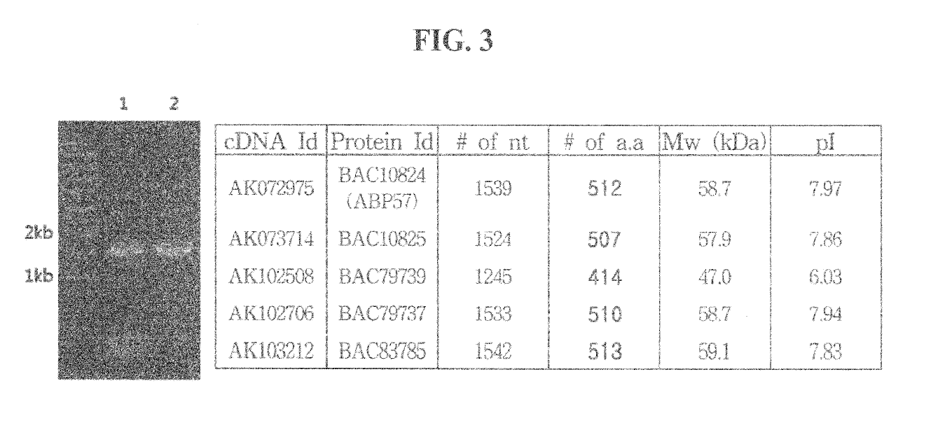

From the stems of rice seedlings of Nakdong variety, which have been grown for 4 weeks to isolate the gene, RNA and genomic DNA were isolated and amplified by RT-PCR. The amplified cDNA fragment (1.5 kb) was cloned into pGEM T-easy vector (Promega), and then the entire base sequencing was carried out. The amplified ABP57 gene encodes the protein of 58.7 kDa, which consists of 512 amino acids with 1,539 by of gene (FIG. 3), and it shows 100% sequence homology with AK072975 registered with NCBI.

### Example 2

### Obtainment of Nucleotide Sequence Information of a Gene which is Analogous to ABP57 and Gene Expression Analysis

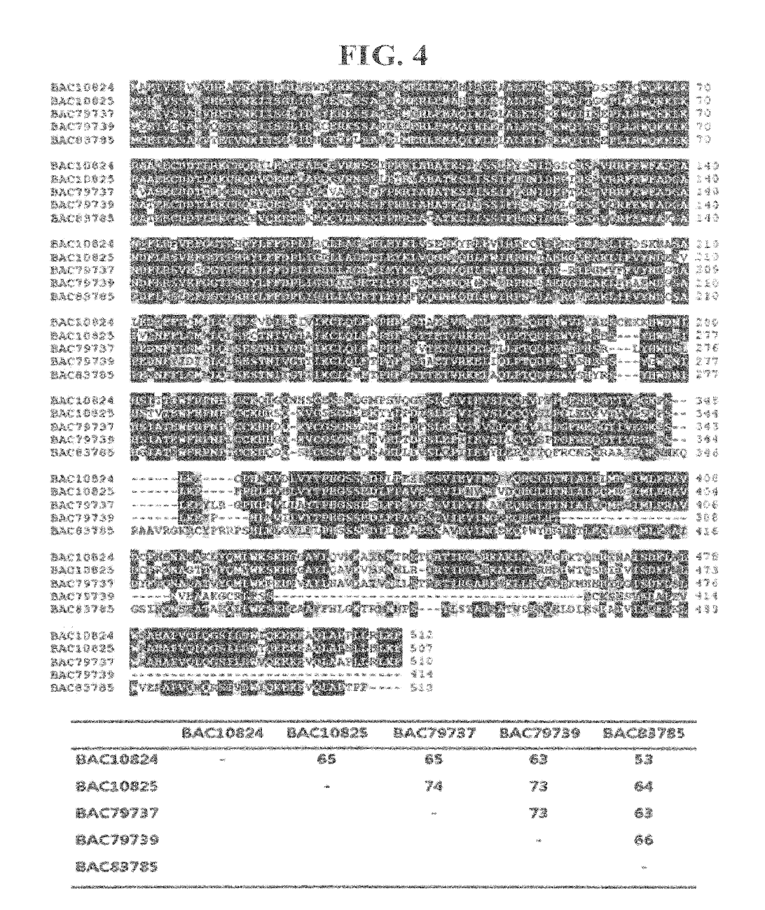

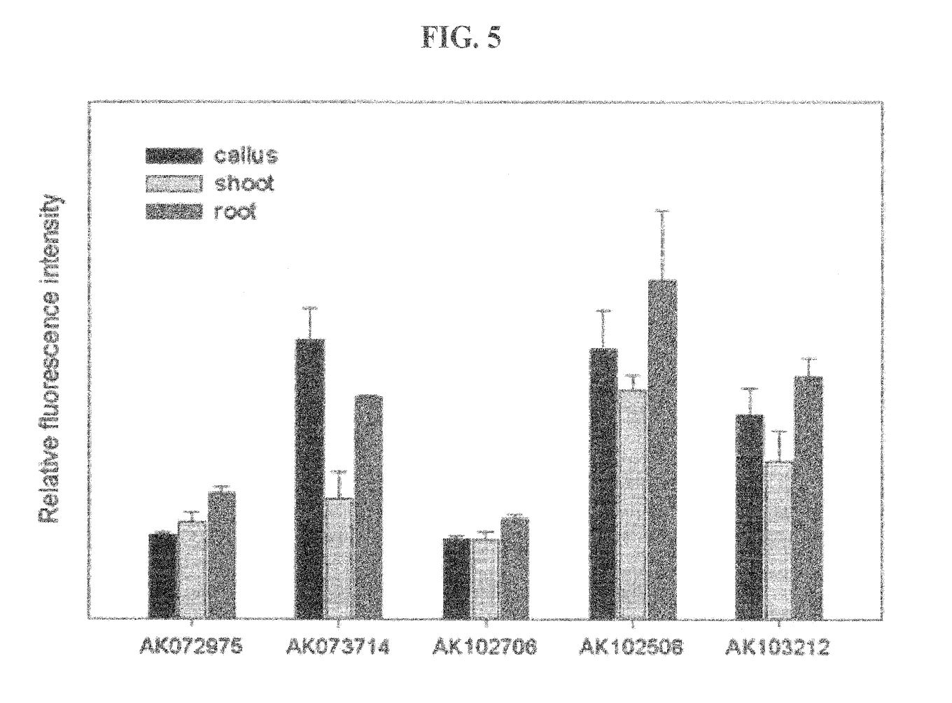

By using the nucleotide sequence information of the gene above, full-length cDNA database search with KOME (Knowledge-based Oryza Molecular Biological Encyclopedia) and EST database search with Maize Genome Sequence Consortium were carried out. As a result, in addition to AK072975, four kinds of analogous genes (AK073714, AK102508, AK102706 and AK103212) were identified from a rice plant, and ORF of each gene shows 53 to 73% of amino acid homology, respectively. Further, one gene with sequence homology to maize was also identified (FIG. 3 and FIG. 4). As a result of rice 60 k microarray analysis, AK072975 and the analogous genes were found to be expressed in callus, stem and roots of a rice plant (FIG. 5).

### Example 3

### Activation of a Proton Pump in Plant Plasma Membrane by ABP57 Recombinant Protein

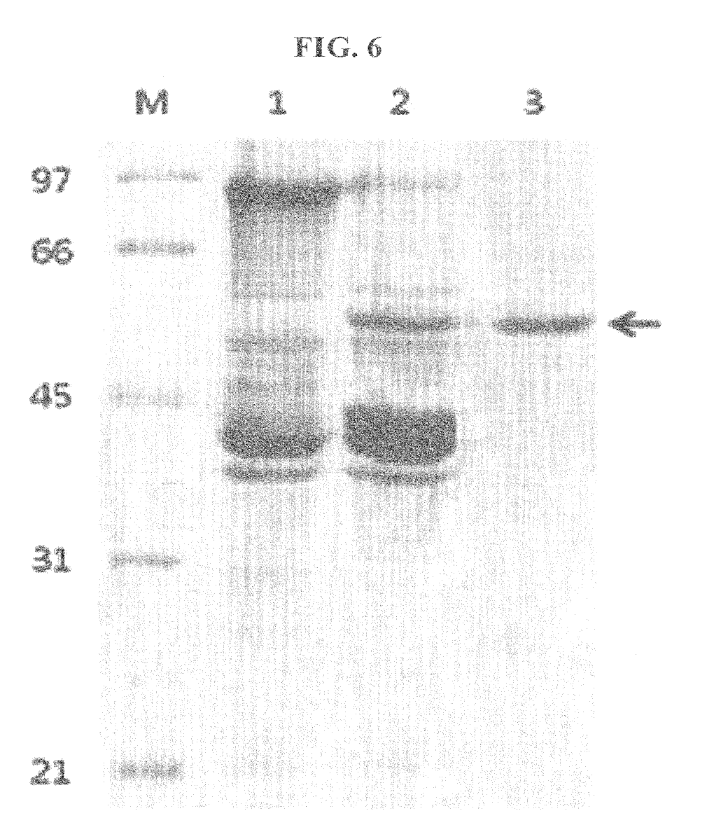

In order to produce ABP57 recombinant protein in E. coli, full-length ORF of ABP57 cDNA was cloned in BamHI and HindIII sites of pMAL-c2x (New England Biolabs). IPTG was added to culture medium for E. coli (E. coli Rosetta(DE3)/pLysS) comprising pMAL-c2x/ABP57 vector to induce the expression of ABP57. By using an amylase resin, MBP-fused ABP57 was purified from the extract of E. coli. To the fused protein, TEV (Tobacco Etch Virus) protease was added (1%) and incubated for 12 hours at 4° C. for a reaction to remove MBP (maltose binding protein) which is bound to ABP57. Finally, the protein was purified by FPLC using HiPrep™ Phenyl FF 16/10 column (FIG. 6).

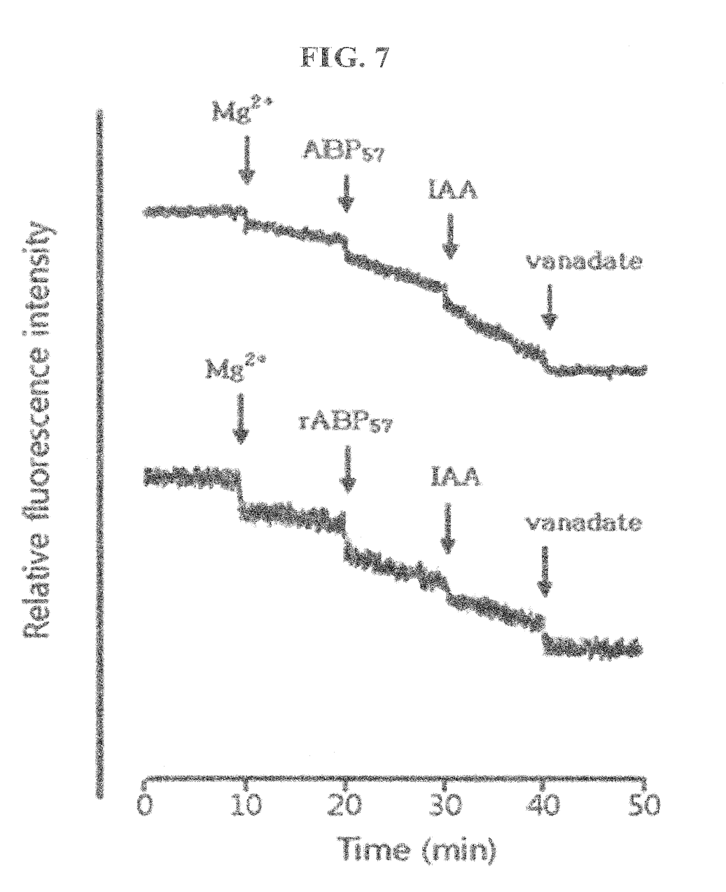

The purified protein and the proton pump in plasma membrane, which had been separated from rice, were admixed with each other, and then the activity of proton pump was measured by fluorescence analyzer. As a result, an increase in the activity of proton pump was confirmed (FIG. 7).

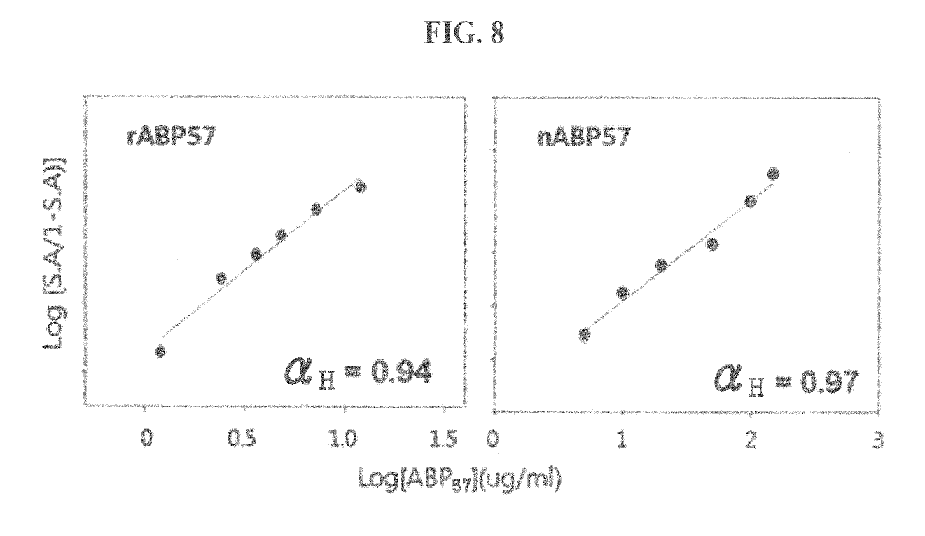

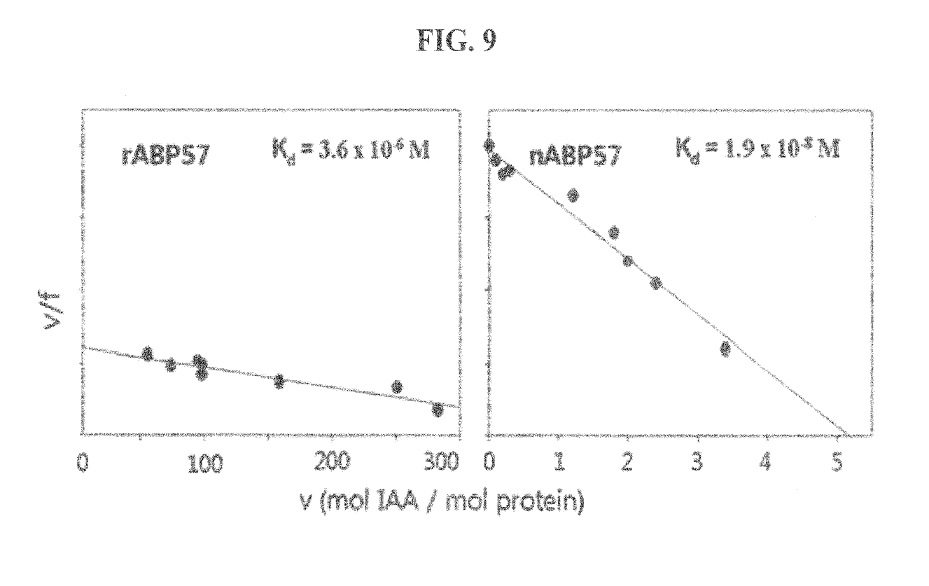

The change in degree of activating proton pump according to concentration of the recombinant protein was measured and analyzed by Hill plot analysis. As a result, the Hill coefficient was found to be 0.94, which is close to the constant 1 indicating the non-cooperative interaction between a protein and a ligand. Further, this value was very close to the Hill coefficient of 0.97, which was obtained by previous studies for the interaction between the proton pump and natural ABP57 (FIG. 8). As a result of Scatchard analysis for determining binding constant between the recombinant protein and auxin hormone IAA, the IAA dissociation constant of the recombinant protein was found to be 3.6×10⁻⁶ M, and the number of binding site was 1, corresponding to the low affinity binding site of natural ABP57 (FIG. 9).

### Example 4

### Determination of the Function of ABP57 by Using a Transformed Plant

In order to amplify ABP57 gene by PCR and construct a plant transformation vector, a pair of PCR primers was produced.

| (SEQ ID NO: 13) | |
|---|---|
| ABP57-5′-1: | 5′-AGTCGGATCCATGGCAGAGA-3′ |
| (SEQ ID NO: 14) | |
| ABP57-3′-l: | 5′-AGTCCTCGAGCTAAAATTTC-3′ |

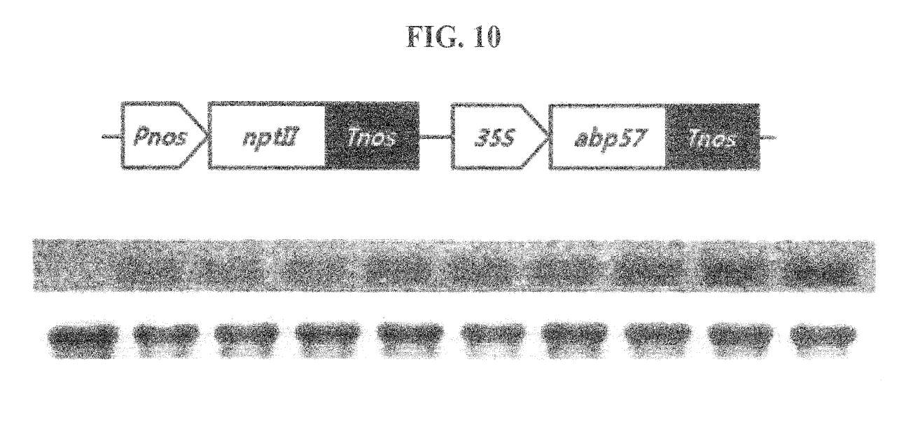

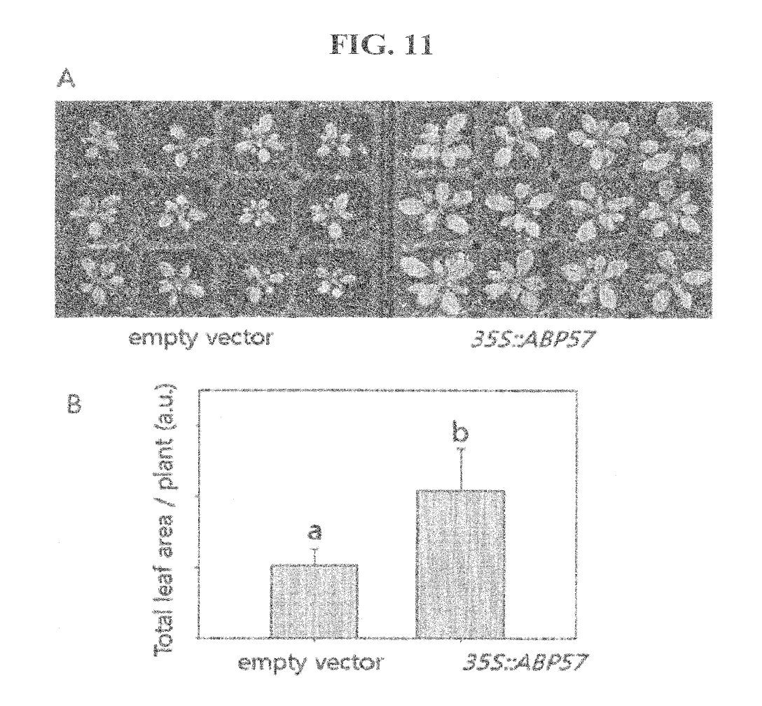

The gene fragment comprising the ORF of the amplified ABP57 was introduced to BamHI and XhoI sites in pBI111L vector (FIG. 10), and it was subsequently introduced to Agrobacterium strain C58C1. Arabidopsis Co1-0 wild type host was transformed by flower dipping method. The seeds harvested therefrom were germinated in a medium comprising kanamycin to select the transformant, which was then transferred to a pot. While growing the plant, expression of the introduced gene was confirmed from the leaves by RT-PCR (FIG. 10). As a result of comparing the growth of the transformant with the control group which had been transformed only with pBI111L vector, it was found that most of the transformants showed growth that is almost two times faster than the control plant (FIG. 11).

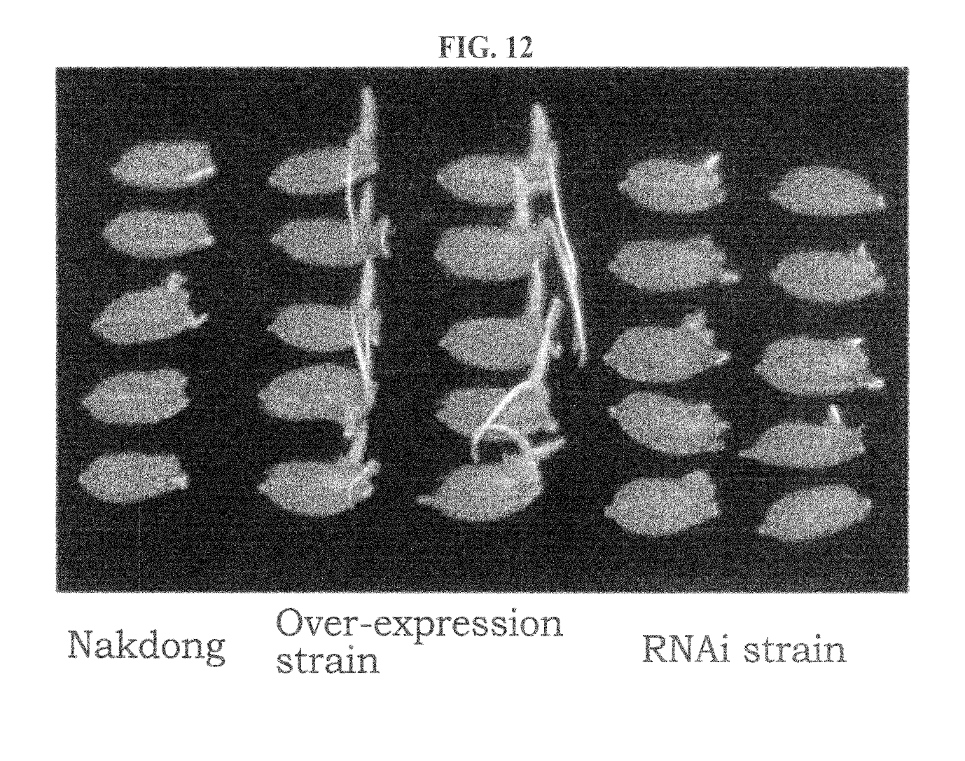

Meanwhile, the rice plant transformed with ABP57 showed promoted germination and early growth compared to the control group (FIG. 12), and the size of the seed was increased as much as 4 to 8% compared to the control group (Table 1).

TABLE 1
Comparison of 100 unpolished grains between the rice
transformed with ABP57 gene and the control group

| | In 100 grains of | Increase ratio in |
|---|---|---|
| Strain | unpolished rice (g) | 100 grains (%) |
| Nakdong (wild type) | 2.166 | 100% |
| 7-14-13 (transformant) | 2.341 | 108% |
| 2-17-14 (transformant) | 2.247 | 104% |

What is claimed is:

1. An auxin receptor protein involved in activation of a proton pump in plasma membrane of a plant derived from rice, wherein the protein has an amino acid sequence selected from a group consisting of amino acid sequence of SEQ ID NO: 1 to 5.

2. A gene which encodes the protein of claim 1.

3. The gene according to claim 2, characterized in that it is selected from a group consisting of nucleotide sequence of SEQ ID NO: 6 to 10.

4. A recombinant vector comprising the gene of claim 2.

5. The recombinant vector according to claim 4, characterized in that it is a recombinant plant expression vector.

6. A host cell transformed with the recombinant vector of claim 4.

7. A method of improving traits of a plant comprising transforming a plant with the recombinant plant expression vector of claim 5.

8. The method according to claim 7, characterized in that the improvement of traits of a plant is a promotion of plant growth.

9. A plant having improved traits by transformation with the recombinant plant expression vector of claim 5.

10. The plant according to claim 9, characterized in that the improvement of traits of a plant is a promotion of plant growth.

11. Seeds of the plant of claim 9.

12. A composition for improving the traits of a plant comprising the gene of claim 2.
